(19) United States
(12) Patent Application Publication
(10) Pub. No.: US 2025/0000719 A1
(43) Pub. Date: Jan. 2, 2025

(54) COVER LAYER FOR A DISPOSABLE HYGIENE ARTICLE, AND DISPOSABLE HYGIENE ARTICLE

(71) Applicant: Nitto Denko Corporation, Osaka (JP)

(72) Inventor: Jan Michael TRINKAUS, Euskirchen (DE)

(73) Assignee: Nitto Denko Corporation, Osaka (JP)

(21) Appl. No.: 18/754,255

(22) Filed: Jun. 26, 2024

(30) Foreign Application Priority Data
Jun. 27, 2023 (DE) 10 2023 116 947.5

(51) Int. Cl. A61F 13/511

(52) U.S. Cl. CPC A61F 13/51121

(57) ABSTRACT

A cover layer for a disposable hygiene article includes a co-extruded laminate film having a first full-area polymer material and a second, elastic polymer material, which is arranged only in certain regions, in the form of at least one strip, to form at least one elastic first functional section with the second, elastic polymer material, and at least one second functional section without the second, elastic polymer material. The co-extruded laminate film has an increased thickness on the at least one strip as compared to the at least one second functional section. The cover layer can be used in a disposable hygiene article such as a diaper.

### CROSS REFERENCE TO RELATED APPLICATIONS

Applicant claims priority under 35 U.S.C. § 119 of German Application No. 10 2023 116 947.5 filed Jun. 27, 2023, the disclosure of which is incorporated by reference.

### BACKGROUND OF THE INVENTION

### 1. Field of the Invention

The invention relates to a cover layer for a disposable hygiene article, in particular a diaper, comprising a co-extruded laminate film having a first full-area polymer material and a second, elastic polymer material, which is arranged merely in certain regions, in the form of at least one strip, to form at least one elastic first functional section with the second, elastic polymer material, and at least one second functional section without the second, elastic polymer material. Another object of the invention is a disposable hygiene article.

### 2. Description of the Related Art

Since elastic components are far more expensive than non-elastic ones, the use of elastic materials and elastic sections is generally restricted to regions that are essential for the comfort of the end user of hygiene products, such as, for example, a side region of a diaper or a center region on the waistband.

In this regard, elastic components are typically glued onto or bonded onto non-elastic components. For example, elastic laminates can be set onto the outer layer of a diaper in order to form diaper ears. In order to minimize the use of elastic materials, it is also known to merely arrange elastic threads or strips of an elastic film in a laminate or to attach them directly to the outer layer.

According to the state of the art, elastic laminates having an elastic film and at least one cover layer composed of a nonwoven are pre-produced, cut to size, and attached to a separately configured diaper body having an outer layer, an inner layer, and an absorbent core arranged between the outer layer and the inner layer.

The methods for affixing such elastic laminates are complicated. The corresponding cut pieces must be cut from an endless web and then precisely arranged on the diaper body and attached. Frequently, this is done with a continuous movement of the diaper bodies, at speeds of about 100 to 600 m/min (meters per minute), wherein then the cut pieces of the elastic laminate must be accelerated and guided accordingly. In addition, affixing of hook-and-loop tapes is also provided as part of a closure system, and this leads to a further increase in complexity.

Incorrect or insufficient attachment of the elastic laminate to the diaper body can lead to the result that the elastic laminate is torn off the diaper body during use, and as a result, the corresponding diaper immediately becomes unusable. If tearing off occurs during use of the diaper, the desired protection is also immediately eliminated. In this regard, it should also be noted that due to production defects or production variations, tearing off can also occur prematurely, before an expected stress resistance is reached.

As has already been explained, comparatively efficient use of elastic materials is possible if merely elastic threads are used to form elastic regions. In the case of these approaches, as well, relatively complicated and error-prone methods occur, in particular because a plurality of threads must be processed at the same time. Furthermore, individual threads or strips can also leave undesirable pressure traces on a user.

Basically, it is known to form laminates having elastic and non-elastic regions by means of co-extrusion. In this regard, a first non-elastic polymer material and a second, elastic polymer material can be arranged next to one another, as strips; in this regard, reference is made to the documents DE 198 06 452 A1, WO 2009/046556 A1, EP 2 411 212 B1, EP 2 533 964 B1.

According to WO 2008/073666 A2, different elastic and non-elastic polymer materials can be arranged in many different ways, for which purpose special extrusion dies are provided. A co-extruded laminate film that is uniform in its external structure can thereby be formed, in its interior, from polymer materials that are different, in certain regions, in order to optimize its function.

From WO 2007/078518 A1, it is known that a polymer material is provided only in certain regions in a co-extruded laminate film, wherein then the interstices are filled up by a further polymer material, which also extends over the aforementioned polymer material with a lesser layer thickness.

The co-extruded laminate films described expand the spectrum of use and allow modulation of the mechanical and, in particular, elastic properties, to a certain degree. Specifically at the transitions between elastic and non-elastic polymer materials, however, there is frequently the risk of tearing.

The measures for producing the known co-extruded laminate films are relatively complicated. Furthermore, the mechanical properties also require improvement with regard to preferred uses in disposable hygiene products.

### SUMMARY OF THE INVENTION

The present invention is based on the object of indicating a cover layer for a disposable hygiene article, which layer can be used in a particularly versatile manner and can simplify the production of a disposable hygiene article. The invention also aims to provide a corresponding disposable hygiene article.

This object is accomplished according to the invention by a cover layer for a disposable hygiene article, in particular a diaper, comprising a co-extruded laminate film having a first full-area polymer material and a second, elastic polymer material, which is arranged, merely in certain regions, in the form of at least one strip for forming at least one elastic first functional section with the second, elastic polymer material, and at least one second functional section without the second, elastic polymer material. According to the invention, it is provided that the co-extruded laminate film has a greater thickness on the at least one strip as compared to the at least one second functional section. It is practical if the thickness ratio is at least 1.2:1.

According to the invention, the at least one first functional section is elastic in the sense of the invention. The at least one second functional section can be both elastic and non-elastic. Within the scope of the invention, embodiments in which the at least one second functional section is non-elastic or at least less elastic than the at least one first functional section are preferred. The second, elastic polymer, which is provided for the good elasticity of the at least one first functional section, is provided only in partial regions, and thereby cost savings can be achieved. Only the first polymer material, which is then non-elastic or less elastic, is arranged over the full area. By means of the different thickness of the co-extruded laminate film, according to the invention, further advantages, explained below, are achieved within the scope of the invention.

According to a further development, it is provided that the ratio of a maximum film thickness determined on the first functional section to a minimum film thickness determined on the second functional section is at least 1.4:1.

According to the invention, it is provided that the co-extruded laminate film has a changing thickness profile. While the first polymer material extends over the full area, the second, preferably elastic polymer material is merely provided in certain sections. There, the additional placement of the second, preferably elastic polymer material results in an increased thickness, wherein outside of the elastic section formed with it, less material, in total, is provided, and this also has a direct effect on the thickness. The at least one second functional section is therefore not filled up with the first polymer material in such a manner that in total, an essentially uniform thickness of the film occurs, as this is known from a different state of the art.

Depending on the configuration, the cover layer can be provided for the purpose of covering a body of the disposable hygiene article essentially completely, from opposite transverse edges and opposite longitudinal edges, or also only in certain regions.

In the case of the cover layer, which is preferably provided as a backing layer, it is provided, according to a further preferred embodiment, that the second polymer material is arranged in the form of at least and particularly preferably precisely two strips that lie adjacent to the longitudinal edges and run in the longitudinal direction. In this regard, it can be provided that in a region of the cover layer that runs in the center along the longitudinal direction, the strips are removed, at least in part, by means of leg cutouts on the sides.

In this regard, it can also be provided that the strips form elastic ears that are integrated into the cover layer, at least in a waist region that follows the center region in the longitudinal direction. Within the scope of the embodiment described, it can fundamentally be provided that the at least one elastic first film section is pre-stretched, completely or in certain regions, i.e. activated, before first-time use, so as to afterwards achieve easy stretching with extensive elastic recovery. Such pre-stretching can be achieved, for example, by means of rolling with rings that engage into one another. Corresponding measures are known to a person skilled in the art.

Furthermore, it is also possible that the cover layer covers only parts, for example ends, viewed in the longitudinal direction, while in between, a center region is formed by a different material.

The co-extruded laminate film of the cover layer can be laminated with a nonwoven, at least in certain regions, so as to form a soft surface. In this regard, the nonwoven can extend, just like the co-extruded laminate film, over the entire surface area of the cover layer, or can be arranged merely in certain regions. In this regard, it can also play a role which regions come into contact with a user, are perceived by a user, or are of importance for the overall impression of the disposable hygiene article.

A further aspect of the invention relates to a disposable hygiene article, in particular a diaper, having the cover layer described above. The disposable hygiene article then preferably has the cover layer as a preferably water-tight outer layer, wherein furthermore, a water-permeable inner layer and an absorbent core arranged between the outer layer and the inner layer are provided. It is also possible that the cover layer forms only part of the outer layer.

According to a preferred embodiment, it is then provided that in the case of the disposable hygiene article in the form of a diaper, the cover layer forms an integrated elastic waistband or diaper ear on the at least one elastic section of the co-extruded laminate film, in a waist region.

In the case of a diaper as a disposable hygiene article, the co-extruded laminate film can be combined, in different ways, with further materials and, in particular, layers of nonwoven, wherein hereinafter, different variants will be discussed as examples in this regard.

Within the scope of the invention, it can be provided that lamination with nonwoven takes place only in elastic sections and, in particular, in a region of diaper ears or elastic waistbands, on one side or both sides. Specifically in elastic sections, a soft surface can be produced by means of a top layer of nonwoven, at least on one side, which layer can also contribute to protection of a user, so as to prevent skin irritation, for example.

Alternatively, another possibility is covering the co-extruded laminate film with a nonwoven over its full area, at least on one side. The connection between the co-extruded laminate film and the nonwoven provided on at least one side can take place in certain sections or over the full area, in this regard.

In the case of coverage with a nonwoven either in certain sections or over the full area, elastic regions can be activated by means of pre-stretching, for example by means of rings that engage into one another, a stretching frame or the like. Extensively complete stretching or also only stretching in certain regions of the co-extruded laminate film can also be achieved by means of two disks set at a slant relative to a transport direction, wherein such a stretching device holds the co-extruded laminate film in a position in which the slanted disks are brought close to one another, and then widening takes place by means of further transport on the disks. If necessary, the first functional sections and the second functional sections can be configured in such a manner that a corresponding force effect stretches essentially the first functional sections, while the tension forces can be absorbed at the second functional sections, without any significant stretching.

Nonwovens that are suitable are, for example, a carded thermobonded nonwoven, a spunbond nonwoven, or also a spunlace nonwoven. Also, different layers of nonwovens can be combined with one another, such as, for example, in the case of an SMS nonwoven (spunbond-meltblown-spunbond).

Strip-wise lamination is also conceivable, wherein the elastic region can be laminated by means of adhesive or ultrasound. The non-elastic region, which is preferably supposed to remain liquid-impermeable, can then preferably be laminated by means of adhesive.

Fundamentally, it is also conceivable to provide a continuous layer of nonwoven on one or both sides, wherein the different types of bonding can then be provided in certain regions, for example by means of adhesive, thermobonding or ultrasound bonding. The type of bonding can then be adapted to the planned function, in each instance, and the stresses to be expected. When using adhesive, it is understood that different bonding patterns and weights per surface area can also be provided in certain regions.

If nonwoven is applied on one side of the co-extruded laminate film, in certain regions, the opposite side can be structured without nonwoven, with full-area nonwoven, or also with nonwoven in certain regions. In the case of placement also in certain regions, the regions on both sides can be arranged to cover one another or to be offset from one another.

Regions of the disposable hygiene article, which is particularly structured as a diaper, can be perforated in order to improve air exchange, for example by means of a laser or vacuum perforation of the co-extruded laminate film. In the case of lamination with at least one nonwoven, such perforation can take place before or after lamination.

In the case of a preferred embodiment, elastic diaper ears or elastic waistband sections are provided in the case of a diaper, on both sides, wherein for this purpose, as described, two elastic film sections of the co-extruded laminate film are provided. Fundamentally, however, it is also possible, alternatively, to lay an elastic film section into the center of a diaper body. If applicable, however, adaptations in the region of an absorbent core are then necessary, so that the function of this core is not impaired in the case of overly strong stretching. It would be conceivable, for example, to refrain from activation in the region of a core, to ensure sufficient strength by means of a further material layer, if necessary, and/or to stretch the co-extruded laminate film during the production process, when laying down the core (corresponding to “stretch-bonding”), so that the core is not subjected to tensile stress during use.

A combination with cover layers composed of nonwoven or the like can also take place on the at least one desired elastic section, in a stretched state (corresponding to “stretch-bonding”). If there is only one first functional section on the diaper, this region is then located on the abdomen and/or the back of a user.

In addition to or as an alternative to activation or stretch-bonding, the co-extruded laminate film can also be combined with materials that are themselves elastic or can be stretched particularly easily, even without activation. Possibilities are, for example, an elastic core and/or an elastic nonwoven as a cover layer.

According to a preferred further development with regard to the co-extruded laminate film, it is provided that the ratio of a maximum total thickness of the first polymer material, determined on the second functional section, to a minimum total thickness of the first polymer material, determined on the first functional section, is at least 2:1.

This aspect relates to the distribution of the first polymer material, which does extend completely, but is provided with a different thickness in certain regions. If, according to a preferred embodiment of the invention, the first polymer material covers the second, elastic polymer material, which is provided as strips, for example, on both sides, the total thickness then relates to the sum of the thickness of the first polymer material, which is present on both sides of the second, preferably elastic polymer material.

Specifically in the case of an elastic configuration of the second polymer material, the particular advantages described below occur. In particular, it can then be provided that the second polymer material is elastic, wherein the first functional section forms an elastic film section and the second functional section forms a non-elastic film section.

In combination with the thickness ratios described above, it is then provided that the first polymer material in the at least one elastic film section is provided with a lesser total thickness than in the non-elastic film section. As will be explained further below, the first polymer material is preferably less elastic, within the scope of the invention, than the second polymer material, wherein the first polymer material, i.e. the layers formed with it, are preferably non-elastic, in total, within the scope of the invention.

The thickness ratio of the first polymer material is therefore inverse to the varying film thickness, according to the embodiment described. With reference to an average weight per surface area or an average total thickness of the first polymer material, the latter is thinned out in the at least one elastic film section.

In this regard, it should fundamentally be noted that then the first non-elastic or less elastic polymer material can lead to increased tensile forces in the at least one elastic film section, and also to greater permanent deformation, something that is generally not desirable. It is true that a thin layer of the first polymer material on the second polymer material, which is elastic according to the preferred embodiment described, is advantageous with regard to laminate strength and frequently also for the placement of further layers such as, for example, layers of nonwoven, but this functionality is already achieved at a low layer thickness. Because of the fact that the first polymer material is present only in a small amount in the at least one elastic film section, the negative influences described are reduced.

Even if the co-extruded laminate film has a lesser film thickness in the non-elastic film section than in the at least one elastic film section, the first polymer material is present there with a greater total thickness than in the at least one elastic film section. There, where the second, elastic polymer material is not present, it cannot contribute to the mechanical stability and strength of the co-extruded laminate film, and for this reason the increased layer thickness of the first polymer material there is advantageous. Taking into consideration the usual properties of elastic and non-elastic polymers as well as the demands on the co-extruded laminate film, the film thickness is nevertheless significantly less than the maximum film thickness determined in the at least one elastic film section, and thereby particularly good and efficient material utilization and cost effectiveness are also achieved. Specifically in the case of disposable hygiene articles, low costs and good material utilization are of particular importance from an economic and ecological point of view.

The co-extruded laminate film is formed, according to a preferred embodiment of the invention, entirely or essentially of the first polymer material and the second polymer material, wherein if necessary, however, thin bonding agent layers introduced during the co-extrusion process, or the like, can also be provided.

According to an alternative embodiment, the co-extruded laminate film comprises a third polymer material, which is provided in the co-extruded laminate film separately from the first polymer material and the second polymer material, as at least one layer.

Preferably, the third polymer material is arranged on the at least one first functional section, between the first polymer material and the second polymer material.

The third polymer material can be used to allow further functions and/or further functional sections in the co-extruded laminate film.

Preferably, the third polymer material is provided everywhere where the second, preferably elastic polymer material is also provided, with reference to a production direction and a transverse direction of the co-extruded laminate film perpendicular to it.

Proceeding from this, the third polymer material can extend over the full area, just like the first polymer material. It is conceivable, for example, that the at least one first functional section is formed with very marked elastic properties, using the second polymer material. Then it is possible that the third polymer material has a lower elasticity than the second polymer material, in order to be able to make certain elastic recovery forces available even outside of the at least one first functional section. In the case of a full-area expanse of the first and third polymer material, these then form the at least one second functional section, which is at least less elastic, depending on its configuration, than the at least one first functional section. The first polymer material can then be provided as a non-elastic outer layer there.

According to a preferred embodiment of the invention, it is provided that the at least one first functional section runs in the form of a strip in the production direction. Such a strip can be achieved, during co-extrusion, in that a melt of the second polymer material is provided only in certain regions, wherein a preferred method for the production of the co-extruded laminate film will be described in detail below.

Within the scope of the invention, it is furthermore preferred if multiple preferably elastic first functional sections are provided, which each form a strip.

At least one strip or preferably at least two strips can have a width, perpendicular to the production direction, between 1 cm and 12 cm, for example. The width of the strips results, in this regard, from the width over which the second, preferably elastic polymer material extends perpendicular to the production direction.

In this regard, it should be noted that the total thickness of the first polymer material, the total thickness of the second polymer material, if applicable the total thickness of the third polymer material, as well as the film thickness, can also change, to a certain degree, over the width of an individual strip.

Usually a certain profile occurs for the aforementioned values, which results from the flow behavior of the polymer materials and, in particular, from their viscosity during the production process. As will still be explained further below, in this connection the melt mass flow rates (MFR) are also significant.

According to a further development of the invention, it is provided that at least two preferably elastic first functional sections in the form of strips run in the production direction, wherein the distance between the strips is between 3 cm and 24 cm. In particular, a plurality of strips can then be provided with a uniform distance or an essentially uniform distance between the strips, so as to form multiple-use material cutouts for disposable hygiene products, for example.

The indicated preferred width of the strips as well as the preferred distance between the strips allows different scaling. For example, such different scaling is practical in order to make available diapers for babies in different age groups or, for example, incontinence articles for adults with different body measurements. In this regard, it can be particularly practical to combine a width in the lower or upper region of the indicated range with a distance that is also in a lower or upper region of the range indicated for this. The ratio of the width of the strips to the distance between the strips and thereby the ratio of the elastic sections to the non-elastic sections can lie, for example, at 1:1 to 1:3.

Within the scope of the invention, the co-extruded laminate film preferably has at least one elastic film section and one non-elastic film section. Within the scope of the invention, a film section is referred to as elastic if, in the case of stretching by 100% with reference to a starting length, recovery by at least 50% with reference to the starting length takes place. Preferably, the elastic section has an elastic recovery of at least 70% after stretching by 100%.

A film section is referred to as being non-elastic, within the scope of the invention, if recovery by less than 50% with reference to the starting length takes place after stretching by 100% with reference to a starting length. Preferably, after stretching by 100%, the recovery is less than 30%.

According to a preferred embodiment of the invention, it is provided that in the at least one first functional section, the second, preferably elastic polymer material is covered on both sides by the first polymer material. In the at least one first functional section, there is then an at least three-layer structure, wherein, in particular, however, thin bonding agent layers can also be provided. For this purpose, the first polymer material can be supplied on both sides as a polymer melt, during production.

In the embodiment described, an essentially uniform structure is then obtained in the at least one non-elastic section, which structure is formed by the first polymer material. If necessary, however, it can still be determined by analysis if the first polymer material is supplied on both sides. This holds true, in particular, if bonding agent layers are provided to increase the laminate adhesion, which layers then also form internal layers or a common internal layer in the non-elastic section.

As explained above, a third polymer material can also be provided. Then preferably a five-layer structure with the second polymer material in the middle, the third polymer material bordering on it on both sides, and finally the first polymer material as layers that lie on the outside is preferably provided in the at least one first functional section.

Within the scope of the invention, it can be provided, in particular, that the co-extruded laminate film has a layer structure that is symmetrical or essentially symmetrical with reference to a center line.

As has also been explained above, the co-extruded film laminate can be formed, in the at least second, preferably non-elastic functional section, entirely or essentially of the first and/or the third polymer material. It is fundamentally preferred that the at least one preferably non-elastic second functional section is formed by at least 70 wt.-% of the first and/or third polymer material. Further functional layers for the purpose of stiffening or the like are preferably not provided. If, within the scope of the variants described, the second functional section is formed by the first and the third polymer material, the first polymer material can be provided on both sides of the third polymer material, in the form of thin outer layers.

The first polymer material, the second polymer material, and, if applicable, the third polymer material can each be formed by a single polymer type having a corresponding product designation. Fundamentally, a blend can be provided for the first polymer material and/or the second polymer material and/or the third polymer material provided, if applicable, wherein different additives and fillers can also be added. The term polymer material therefore refers, in this regard, to the polymer or polymer mixture extruded from the melt, including any additives.

According to a preferred embodiment of the invention, the first polymer material has a polyolefin or multiple polyolefins as the main polymer component. Aside from polymer components, fillers can also be used, which can be provided at different weight proportions, depending on the case of use. Fillers can contribute to improving the properties and/or to reducing the costs, depending on the requirements. In particular for use in a disposable hygiene article, for example as the back layer of a diaper, talcum can be provided. Talcum as the pulverized form of the mineral talc belongs to the group of layered silicates, wherein fundamentally, other layered silicates can also be used as fillers within the scope of the invention. Talc is inexpensive and, as a layered silicate, can contribute to improving the mechanical properties. Furthermore, talc can be used to achieve greater breathability after stretching, wherein certain pores and openings can occur at the particles of the layered silicate. In order to achieve particularly great activity, blends having a high proportion of chalk are provided, wherein then particularly many pores can be produced by means of stretching.

If layered silicates or other fillers are provided, then these can be present, for example, at a proportion of 4 wt.-% to 70 wt.-%, in particular 5 wt.-% to 20 wt.-%. A particularly high proportion of more than 40 wt.-% is provided, in particular, when chalk is the filler.

Aside from such fillers, other additives such as pigments, UV-stabilizers, processing aids or the like can also be provided. Corresponding organic and/or preferably inorganic functional additives are preferably provided at a proportion of less than 10 wt.-%, in particular less than 5 wt.-%.

Polyethylenes and polypropylenes, in particular, are suitable as the polymer component of the first polymer material.

As has already been explained above, the second functional sections, in particular, are formed with the first polymer material, in some of the embodiments described, in their entirety or to a large proportion. Although the second functional sections, as defined above, preferably do not have any marked elastic properties, a certain admixture of 3 wt.-% to 30 wt.-%, for example, in particular 5 wt.-% to 20 wt.-% thermoplastic polyolefin elastomer (TPO) can be practical for the first polymer component. These proportions are not provided for the purpose of and also not sufficient for imparting elastic properties to the corresponding regions, in the sense of the invention. Orientation experiments have shown, however, that the risk of tearing under tensile stress can be significantly reduced, in part, at the transitions between the at least one preferably elastic first functional section and the at least one preferably non-elastic second functional section.

The second, preferably elastic polymer material can contain at least one styrene block copolymer and/or at least one polyolefin elastomer. The suitable styrene block copolymers include, for example, styrene butadiene styrene (SBS), styrene isoprene styrene (SIS), styrene ethylene butadiene styrene (SEBS) and styrene ethylene propylene styrene (SEPS). According to a preferred embodiment, the first polymer material has 60 to 90 wt.-% SBS. For example, SBS types having a styrene/butadiene ratio in the range of 20:80 to 40:60, for example 30:70, are suitable.

If the third polymer material is provided, a polyolefin elastomer (TPE-O) is preferred for this purpose. Preferably, then, a combination with the first non-elastic polymer material on the basis of non-elastic polyolefin and the second polymer material on the basis of a styrene block copolymer takes place.

According to the invention, the ratio of the maximum film thickness determined in the at least one first functional section to the minimal film thickness determined in the at least one second functional section is at least 1.2:1. The ratio usually lies in a range between 1.4:1 and 3:1, in particular in a range between 1.5:1 and 2:1.

The maximum total thickness can be, for example, between 25 μm and 75 μm, in particular between 30 μm and 50 μm.

The minimum total thickness can be, for example, between 10 μm and 40 μm, in particular between 19 μm and 31 μm.

Preferably, it is provided that the ratio of the maximum total thickness of the first polymer material determined in the second functional section to the minimum thickness of the first polymer material determined in the first functional section is at least 2:1. Particularly preferably, the ratio is at least 3:1. Usually the ratio lies in a range between 2:1 and 9:1.

Also with regard to a possible production method according to the invention, the melt mass flow rates of the first polymer component and of the second, elastic polymer component are relevant, as is their ratio, in particular.

According to a preferred embodiment of the invention, it is provided that the ratio of the melt mass flow rates (MFR) at 200° C. and 5 kg of the first polymer material and of the second polymer material is 1:1 to 2:1. The melt mass flow rates relate, in this regard, to the first and the second polymer material, in other words—as explained above—to the polymer or polymer mixture extruded from the melt, including any additives.

The definition of the melt mass flow rates refers, in this regard, to the standard ASTM D1238 in the version in effect on the application date, taking into consideration the test temperature of 200° C. and the test load of 5 kg that are indicated in concrete terms.

For the second polymer material, the melt mass flow rates can lie, for example, in a range from 14 to 27 g/10 min (200° C./5 kg), wherein then the melt mass flow rates of the first polymer material occur on the basis of the ratio indicated above.

If the third polymer material is provided, the ratio of the melt mass flow rates (MFR) at 200° C. and 5 kg of the third polymer material and of the second polymer material is preferably 2:1 to 1:2, in particular 1:1 to 2:1.

The co-extruded laminate film can fundamentally be formed in different ways. In this regard, it is essential to the invention that the described ratios of the film thickness as well as of the first polymer material are achieved with reference to the elastic section and the non-elastic section.

Fundamentally, it is conceivable to provide special contouring in an extrusion die, at the exit gap, and/or to supply the polymer materials in a suitable manner within the co-extrusion die.

In a preferred embodiment of the method, a melt of the first polymer material is guided over the full area, and a melt of the second polymer material is guided merely in the form of at least one strip, in an extrusion die, wherein the first polymer material and the second polymer material are extruded jointly, along a production direction, using the extrusion die, in such a manner that the co-extruded laminate film is formed with a varying thickness profile along a transverse direction.

The co-extruded laminate film is formed directly in a uniform co-extrusion process. In order to achieve the thickness ratios as described in this regard, different measures and properties can be utilized and also combined with one another. As will also be explained further below, a die gap, in particular a cast die, can be permanently pre-formed at its exit, in such a manner that the desired profiling of the co-extruded laminate film itself is at least partially predetermined by means of profiling of the gap, with narrow locations and widened parts. In this regard, the profiling must be coordinated with the feed of the first polymer material and of the second polymer material, which is merely provided in certain sections, and it has then been established, to a great extent.

In addition or alternatively, the co-extrusion can also be carried out using a cast die, which has an adjustable die lip. While the die lip is usually used, in the state of the art, to achieve a uniform film thickness over a film width, the die lip can be used in a targeted manner, within the scope of the invention, so as to predetermine or support the profiling of the co-extruded laminate film, according to the invention, at least in part. Also, in this regard, the setting of the die lip must be coordinated with the feed of the first polymer material and of the second polymer material, which is provided merely in certain sections. In the case of a change of the width and/or of the distance between strips of the second polymer material, for example, the die lip can then be adjusted in a suitable manner.

During co-extrusion, widening of the melt occurs as it leaves the extrusion gap, due to the pressure drop that is connected with it; this is also referred to as extrudate swell. In this connection, a different melt elasticity of the first polymer component and of the second polymer component can also be utilized so as to obtain the desired profiling of the co-extruded laminate film, at least in part. Also in the case of the measures mentioned previously, as examples, the melt elasticity should at least be taken into consideration for practical purposes.

According to a concrete embodiment, three coat-hanger manifolds can be provided for a three-layer structure, on the at least one first functional section, on the cast die, arranged one on top of the other, wherein the second polymer material is supplied to the center coat-hanger manifold. The center coat-hanger manifold has retaining elements for reducing the total melt stream of the second polymer material to the strips.

At the end of the cast die, the die exit cross-section is then reduced at the at least one second functional section. As described above, such a reduction can be achieved by means of a reduction in the channel cross-section up to the die gap and/or the adjustable die lip. The adjustment of the die lip can be made possible by means of setting screws or other setting elements, wherein fundamentally also a drive, a controller and/or a regulator of such setting elements is also possible. For example, properties of the co-extruded laminate film, such as the location of the functional regions, thicknesses, or the like, can also be detected and used for control and/or regulation.

As described above, it is possible to produce the strips directly in a coat-hanger manifold. Fundamentally, however, it is also possible to already form strips of the second polymer material previously, in a co-extrusion adapter, wherein then a melt of the full-area first polymer material and a melt of the second polymer material that is merely provided in the form of strips are then expanded, for example in a coat-hanger manifold of a cast die. The measures described above for contouring the co-extruded laminate film at the die gap are then also provided.

Within the scope of the invention, the co-extruded laminate film is subjected to stress with at least one preferably elastic first functional section and at least one preferably non-elastic second functional section. If, according to a preferred embodiment of the invention, multiple first and/or multiple second functional sections are provided, then these are preferably structured as described above, in each instance. In particular, it can be provided that the first and/or the second functional sections is/are structured or at least designed to be the same or essentially the same, in each instance, relative to one another. In this regard, usual production variations should not be taken into consideration.

Taking concrete requirements into consideration, however, it is also possible to structure specific preferably elastic first functional sections and/or preferably non-elastic second functional sections differently.

With regard to a preferred use of the co-extruded laminate film for disposable hygiene articles, the co-extruded laminate film can be produced with a large width of more than a meter, for example, wherein then this co-extruded laminate film is cut for multiple uses. In particular, individual strip-shaped webs for further processing can then be cut from such a wide co-extruded laminate film having a plurality of preferably elastic first functional sections and preferably non-elastic second functional sections.

In the following Table 1, exemplary embodiments 1 to 5 are shown.

TABLE 1

| | | | Maximum | Minimum | Thickness |
|---|---|---|---|---|---|
| | Second polymer material | First polymer material | thickness [μm) | thickness [μm] | ratio |
| 1 | 75 wt.-% SBS with styrene/ | 100 wt.-% PE-LLD | 46 | 30 | 1.5 |
| | butadiene ratio of 30:70 | MFR | | | |
| | 12 wt.-% PS | (200° C./5 kg): 20.9 g/10 min | | | |
| | 8 wt.-% plasticizer | | | | |
| | 5 wt.-% PE, PE-copolymers | | | | |
| | MFR (200° C./5 kg): 15.2 g/10 min | | | | |
| 2 | 75 wt.-% SBS with styrene/ | 60 wt.-% PE-LLD | 43 | 27 | 1.6 |
| | butadiene ratio of 30:70 | 32 wt.-% PP | | | |
| | 12 wt.-% PS | 8 wt.-% talcum | | | |
| | 8 wt.-% plasticizer | MFR | | | |
| | 5 wt.-% PE, PE-copolymers or | (200° C./5 kg): 27.0 g/10 min | | | |
| | combinations | | | | |
| | MFR (200° C./5 kg): 15.2 g/10 min | | | | |
| 3 | 75 wt.-% SBS with styrene/ | 60 wt.-% PE-LLD | 39 | 20 | 2.0 |
| | butadiene ratio of 30:70 | 32 wt.-% PP | | | |
| | 12 wt.-% PS | 8 wt.-% talcum | | | |
| | 8 wt.-% plasticizer | MFR | | | |
| | 5 wt.-% PE, PE-copolymers | (200° C./5 kg): 27.0 g/10 min | | | |
| | MFR (200° C./5 kg): 15.2 g/10 min | | | | |
| 4 | 75 wt.-% SBS with styrene/ | 80 wt.-% PP-BC | 37 | 19 | 1.9 |
| | butadiene ratio of 30:70 | 20 wt.-% TPE-O | | | |
| | 12 wt.-% PS | MFR | | | |
| | 8 wt.-% plasticizer | (200° C./5 kg): 14.3 g/10 min | | | |
| | 5 wt.-% PE, PE-copolymers | | | | |
| | MFR (200° C./5 kg): 15.2 g/10 min | | | | |
| 5 | 75 wt.-% SBS with styrene/ | 53 wt.-% PE-LLD | 32 | 13 | 2.5 |
| | butadiene ratio of 30:70 | 32 wt.-% PP | | | |
| | 12 wt.-% PS | 8 wt.-% TPE-O | | | |
| | 8 wt.-% plasticizer | 7 wt.-% talcum | | | |
| | 5 wt.-% PE, PE-copolymers | MFR | | | |
| | MFR (200° C./5 kg): 15.2 g/10 min | (200° C./5 kg): 23.9 g/10 min | | | |

In all the exemplary embodiments, styrene-butadiene-styrene (SBS) is provided as the second polymer material, at 75%, while the remainder is formed by polystyrene (PS) with the usual admixtures and fillers. The properties of the second polymer material are determined, in this regard, essentially by the SBS, and the usual admixtures and fillers are known to a person skilled in the art. The styrene/butadiene ratio is 30:70, wherein the block copolymers are preferably symmetrical or essentially symmetrical. Corresponding SBS types can be obtained, for example, from the companies Kraton Dynasol and TSRC.

In the case of the first exemplary embodiment, the first polymer material is formed, at 100 wt.-%, from linear low-density polyethylene (PE-LLD) having the properties indicated. Corresponding PE types can be obtained, for example, from the manufacturers Dow (Dowlex), Argus (Argutex), Borealis, and Exxon.

According to the second exemplary embodiment, in the case of the first polymer material, the PE-LLD is provided at a proportion of 60 wt.-%. Furthermore, the first polymer material contains polypropylene (PP) and talcum, as indicated.

The second and third exemplary embodiments differ essentially in terms of the maximum thickness and the minimum thickness of the co-extruded laminate film.

In the case of the fourth exemplary embodiment, polypropylene block copolymer (PP-BC) having the listed properties is mixed with a thermoplastic elastomer on an olefin basis. The polypropylene block copolymer can be obtained from Dow (Dowlex), Argus (Argutec), Borealis or Exxon, for example. The thermoplastic elastomer on an olefin basis can be obtained from Exxon (Vistamaxx) or Dow (Infuse), for example.

With regard to the exemplary embodiments, elastic properties are shown for a strength tensile test and a hysteresis measurement of the elastic properties, in Tables 2 and 3. The measurements relate to an analysis in the transverse direction, in other words perpendicular to the production direction.

TABLE 2

| | | Strength | | | | | |
|---|---|---|---|---|---|---|---|
| | | | Stretching | F- | F- | Stretching | Stretching |
| | Measurement | | at rupture | 10% | 100% | at 5N | at 10N |
| Example | position | Fₘₐₓ-[N] | [%] | [N] | [N] | [%] | [%] |
| 1 | elastic section | 22.1 | 818.1 | 3.1 | 4.3 | 232.5 | 615.7 |
| 2 | elastic section | 31.3 | 893.0 | 4.4 | 5.6 | 20.8 | 503.9 |
| 3 | elastic section | 28.9 | 975.0 | 3.1 | 4.2 | 260.4 | 623.5 |
| 4 | elastic section | 33.6 | 946.9 | 3.9 | 5.3 | 47.0 | 512.4 |
| 5 | elastic section | 7.4 | 575.7 | 2.8 | 3.8 | 356.3 | — |
| 1 | non-elastic section | 23.6 | 1069.8 | 6.6 | 6.6 | 4.8 | 632.4 |
| 2 | non-elastic section | 16.5 | 724.9 | 9.5 | 9.1 | 1.7 | 156.1 |
| 3 | non-elastic section | 7.1 | 577.1 | 4.7 | 5.0 | 40.8 | — |
| 4 | non-elastic section | 16.2 | 817.9 | 6.2 | 6.1 | 4.6 | 546.7 |
| 5 | non-elastic section | 7.8 | 802.5 | 3.5 | 3.7 | 540.3 | — |

TABLE 3

| | | Hysteresis | | | | | |
|---|---|---|---|---|---|---|---|
| | | Force- | Force- | Force | Force | Force | |
| | Measurement | 50% | 100% | 120% | 50% U2 | 75% U2 | |
| Example | position | [N] | [N] | [N] | [N] | [N] | P-Set[%] |
| 1 | elastic section | 4.2 | 4.3 | 4.3 | 0.6 | 1.3 | 14.6 |
| 2 | elastic section | 5.4 | 5.6 | 5.6 | 0.6 | 1.1 | 15.3 |
| 3 | elastic section | 3.9 | 4.1 | 4.1 | 0.5 | 1.0 | 16.5 |
| 4 | elastic section | 5.0 | 5.3 | 5.3 | 0.4 | 0.9 | 17.7 |
| 5 | elastic section | 3.6 | 3.8 | 3.7 | 0.4 | 0.9 | 17.8 |

The strength tensile test takes place by analogy to DIN ISO 527 (1 to 3). In the tensile test, the material properties are determined under single-axis tensile stress and with an initially uniform tension distribution over the cross-section. Corresponding testing devices are available from the Zwick company. The testing takes place in a normal climate, wherein samples having a width of 25.4 mm are made available. The clamping length of the samples is 30 mm. Testing takes place proceeding from a preload of 0.1 Newton. The preload ensures that the sample is correctly clamped in place and held in place in a force-free manner, to a great extent. The speed up to the set preload is 30 mm/min. Testing is then carried out at a measurement speed of 50 mm/min up to stretching of 100%, and 500 mm/min starting from 100% stretching.

The hysteresis measurement takes place using a Zwick Z010 testing device, at a temperature of 23° C.±2° C. and a relative humidity of 50%±5%. The sample width and the clamping length are 25.4 mm, in each instance, wherein a preload is set to 0.05 Newton for the beginning of the measurement. The speed up to the set preload is 50 mm/min. The measurement speed is then set to 500 mm/min for the measurement. During the measurement, two cycles are passed through, wherein the lower reversal point is set to 121% stretching with reference to the starting length. The holding time in the stretched state is 30 seconds. The holding time in the relaxed state between the two cycles is 60 seconds. The values indicated in Table 2 and 3 are generated and output by the test device. The values in the table that are marked with the addition U2 refer to the elastic recovery after the second stretching (“unload 2”).

### BRIEF DESCRIPTION OF THE DRAWINGS

Other objects and features of the invention will become apparent from the following detailed description considered in connection with the accompanying drawings. It is to be understood, however, that the drawings are designed as an illustration only and not as a definition of the limits of the invention.

In the drawings,

FIG. 1 shows a co-extruded laminate film,

FIG. 2 shows a co-extrusion adapter for the production of the co-extruded laminate film,

FIG. 3A shows a partial view of an extrusion die for the production of the co-extruded laminate film,

FIG. 3B shows a further partial view of the extrusion die for the production of the co-extruded laminate film,

FIG. 4 shows a top view of a co-extruded laminate film with a cutting pattern for forming cover layers for a disposable hygiene article,

FIG. 5 shows a cover layer formed from the co-extruded laminate film,

FIG. 6 shows an alternative embodiment of a co-extruded laminate film and a pre-product for a disposable hygiene article with the co-extruded laminate film,

FIG. 7 shows a further alternative embodiment of a co-extruded laminate film,

FIG. 8 shows a disposable hygiene article, and

FIG. 9 shows a force/stretching diagram.

### DETAILED DESCRIPTION OF PREFERRED EMBODIMENTS

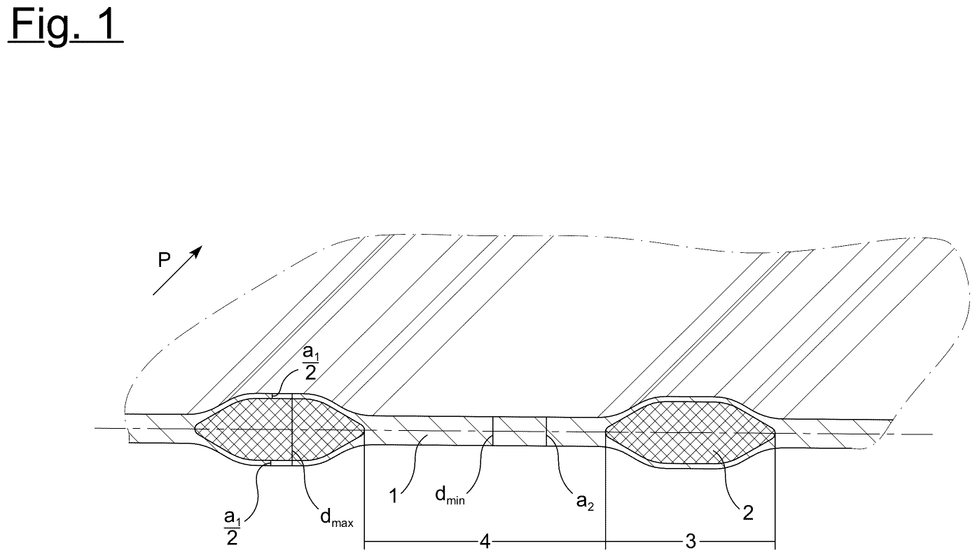

FIG. 1 shows a co-extruded laminate film having a first polymer material 1 and a second, elastic polymer material 2, wherein the first polymer material 1 extends over the full area and wherein the second, elastic polymer material 2 is provided merely in certain sections. The second, elastic polymer material 2 extends in the form of strips along a production direction P, wherein elastic first functional sections 3 with the second polymer material 2 and, in the exemplary embodiment shown, preferably non-elastic second functional sections 4 without the second polymer material 2 are formed.

The ratio of a maximum film thickness dₘₐₓ determined in the first functional sections 3 to a minimum film thickness dₘᵢₙ determined in the second functional sections 4 is then at least 1.4:1. The ratio of a maximum total thickness a₂ of the first polymer material 1 determined in the second functional sections 4 to a minimum total thickness a₁ of the first polymer material 1 determined in the first functional sections 3 is at least 2:1.

In this regard, it can be recognized that there is symmetry with reference to a center plane of the co-extruded laminate film, wherein a symmetrical three-layer structure occurs in the first functional sections 3. There the second, elastic polymer material 2 is covered, in each instance, with a layer having a thickness of a₁/2 of the first polymer material 1.

The strips that run along the production direction P, i.e. corresponding to the first functional sections 3, have a width between 1 cm and 12 cm perpendicular to the production direction P. The distance between two first functional sections 3 and thereby the width of the second functional sections 4 is between 3 cm and 24 cm.

While a three-layer structure occurs in the first functional sections 3, in the exemplary embodiment shown, the second functional sections 4 are formed exclusively by the first polymer material 1.

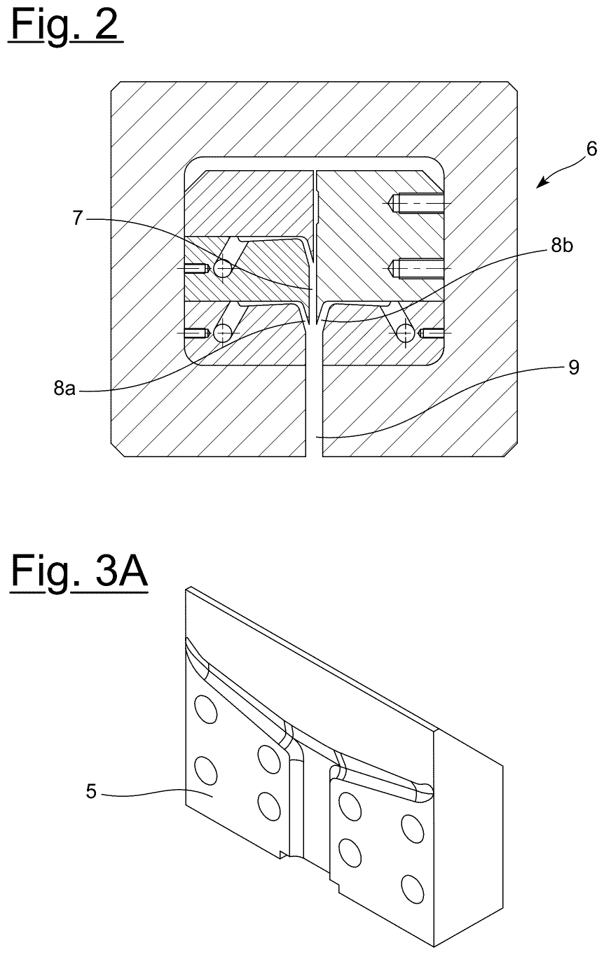

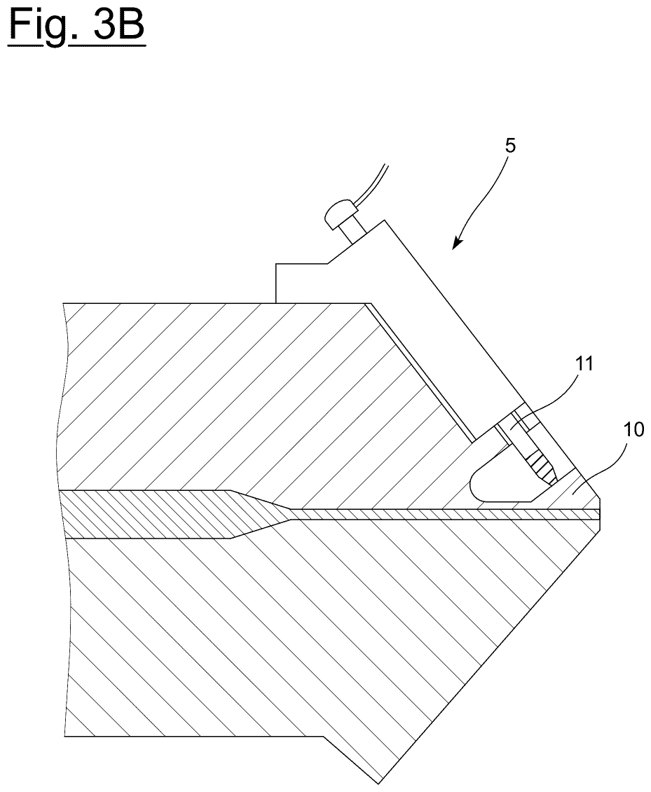

The co-extruded laminate film can be produced in that a melt of the first polymer material 1 is supplied over the full area, and a melt of the second polymer material 2 is supplied merely in certain regions, in the form of strips, to an extrusion die 5 shown as an example in a partial view in FIG. 3A, for example in the form of a cast die having a co-extrusion adapter 6 (feed block) shown in FIG. 2. According to FIG. 2, the second polymer material 2 is supplied in the form of strips, in a center feed gap 7, while the first polymer material 1 is introduced through feed gaps 8a, 8b that lie on the outside, into a melt channel 9 within the co-extrusion adapter 6.

The first polymer material 1 is introduced by way of the feed gap 8a, 8b that lies on the outside.

In the extrusion die 5, the melt strand is widened to form the co-extruded laminate film. In order to achieve the thickness profile shown in FIG. 1, a die lip 10 can be provided on the extrusion die 5, as shown in FIG. 3B, which lip can be adjusted by means of a plurality of setting means 11.

In the exemplary embodiment shown as an example, the strips are already formed in the co-extrusion adapter 6. Alternatively, an extrusion die 5 having three coat-hanger manifolds can also be provided, for example, wherein the second polymer material 2 is supplied to the center coat-hanger manifold. The center coat-hanger manifold then has retaining elements for reducing the total melt stream of the second polymer material to the strips.

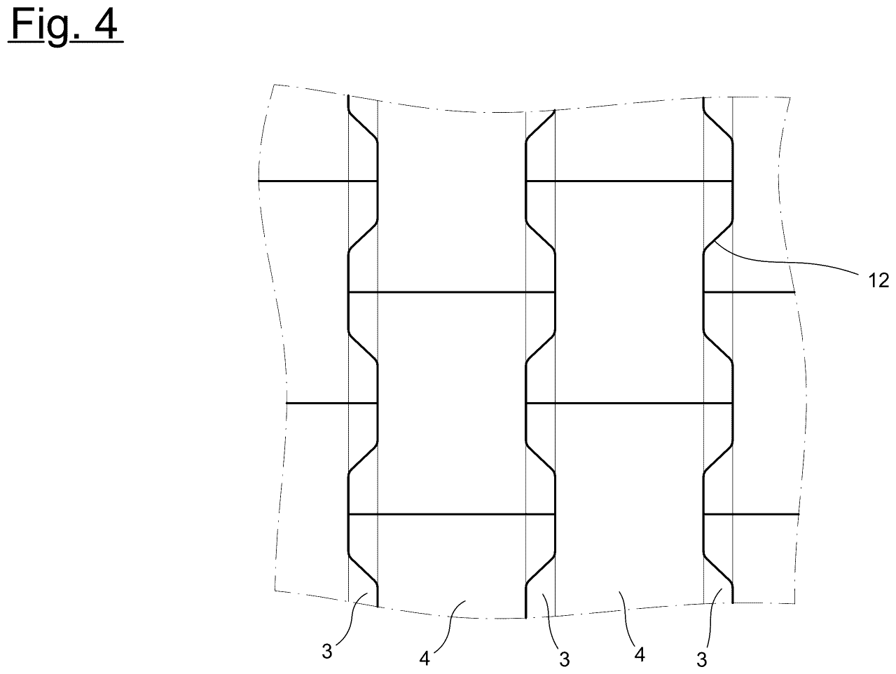

FIG. 4 shows a top view of the co-extruded laminate film with a plurality of elastic sections 3 and non-elastic sections 4. Furthermore, cutting lines 12 are included in the drawing, so as to form cover layers for a disposable hygiene article, in particular a diaper, from the co-extruded laminate film.

The cutting lines 12 are arranged in such a manner that each cover layer 2 has elastic sections 3 at its edges, which are removed in a center region, in each instance, by means of leg cutouts on the sides.

In this regard, the elastic sections 3 form elastic ears integrated into the waist regions of the cover layer that follow the center region in the longitudinal direction. To form a pull-up diaper, the sections can be connected to one another, for example bonded or glued. In order to form a diaper that can be put on using closure means, such closure means, for example hook-and-loop closures, can be placed on the integrated elastic ears.

To form the entire cover layer, the co-extruded laminate film can be laminated to at first one nonwoven, at least in certain regions.

In this regard, the placement of a nonwoven can take place over the full area, so that then the cover layer shown in FIG. 4 has at least one layer of nonwoven over the full area, on one side or also on both sides. Fundamentally, however, it is also possible to arrange nonwoven only partially on one side or on both sides, so as to cover specific functional regions, in particular functional regions that are of particular importance for the haptics, the appearance and/or the function of the cover layer.

The co-extruded laminate film can be pre-stretched, at least in certain regions. In the region of the non-elastic sections, pre-stretching can take place, for example, so as to achieve breathability. This is possible, for example, if the first polymer material is provided with fillers that form pores during stretching. In particular, it is thereby possible to configure the cover layer, as an outer layer of the diaper, to be both water-tight and breathable.

Pre-stretching in the first functional sections 3, also referred to as activation, can be practical in order to afterwards achieve easy stretchability during use of the diaper.

In the case of a disposable hygiene article, in particular in the form of a diaper, the cover layer can therefore be provided as a water-tight outer layer and be combined with a water-permeable inner layer. In this regard, an absorbent core is usually arranged between the water-permeable inner layer and the outer layer.

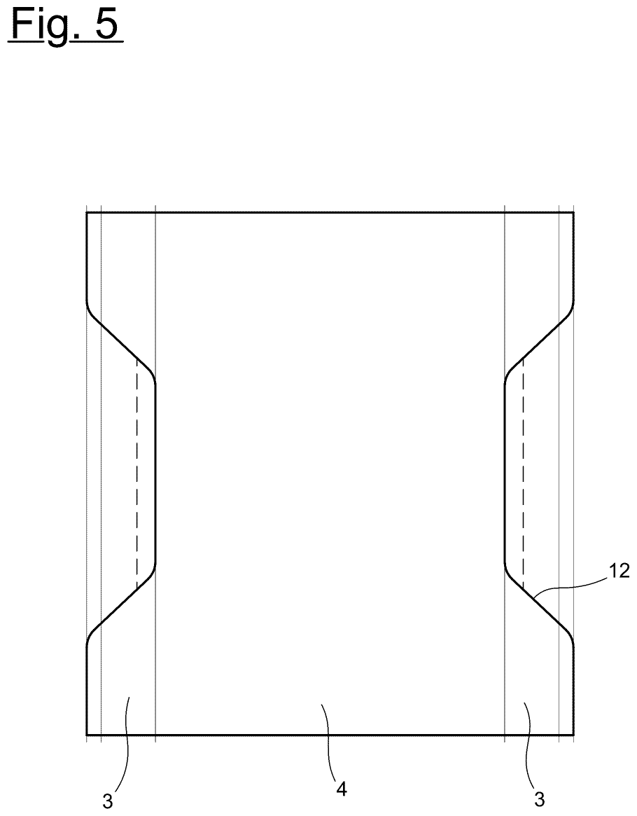

FIG. 5 shows, in a further embodiment, a cover layer that is cut from a co-extruded laminate film. In this regard, it is also indicated that the cutting lines 12 can run in such a manner that a strip of the first functional section 3 still remains on the lateral leg cutouts, wherein there then, too-if necessary after activation-certain elastic reset properties can be achieved, so as to achieve a better fit on the legs of a wearer.

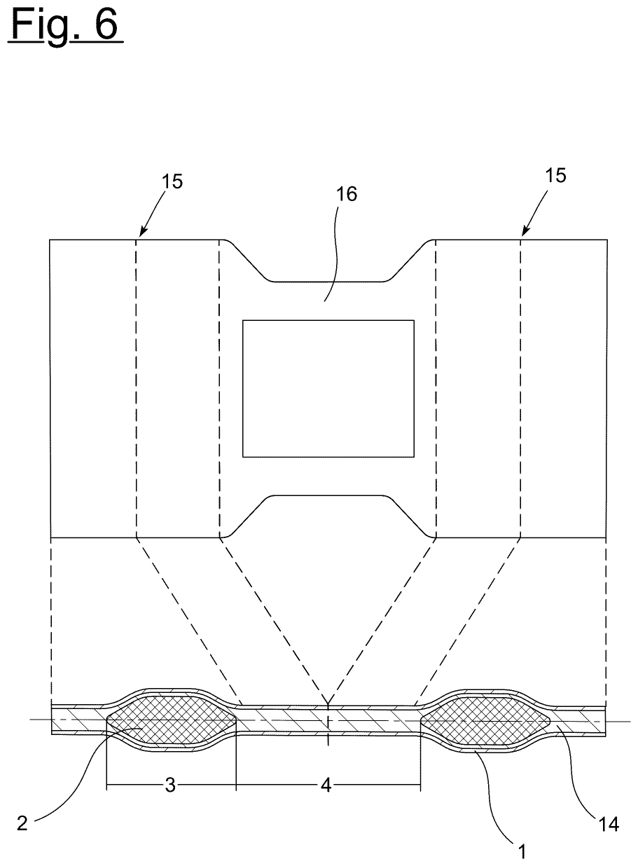

FIG. 6, for a better understanding, shows an alternative embodiment of the co-extruded laminate film in one figure, on the one hand, and a pre-product formed with it, for a disposable hygiene article 13 having the co-extruded laminate film. The co-extruded laminate film, proceeding from the embodiment according to FIG. 1, has a third polymer material 14, which, like the first polymer material 1, extends over the full area. In the first functional sections 3, a structure having five layers is obtained. By means of the second polymer material 2, the first functional sections 3 have great elasticity. The third polymer material 14 also still has certain elastic properties, but a worse recovery capacity than the second polymer material. In this regard, the second functional sections 4 can still be elastic, in the sense of the invention, depending on their configuration. For a distinction from the first functional sections 3, the second functional sections 4 can also be referred to as semi-elastic.

The co-extruded laminate film according to FIG. 6 can be used to make regions having different elasticity available in the disposable hygiene article 13 in the form of a diaper. For this purpose, the co-extruded laminate film can be cut and arranged as shown, so that the first and second functional sections 3, 4, which run in the production direction in the co-extruded laminate film, run along transverse edges in the pre-product. Using the cutout of the co-extruded laminate film that is shown and cut through in its center, a front and a back waistband region 15 of the disposable hygiene article 13 can be formed, wherein a center section 16 having an absorbent core is formed by a separate material.

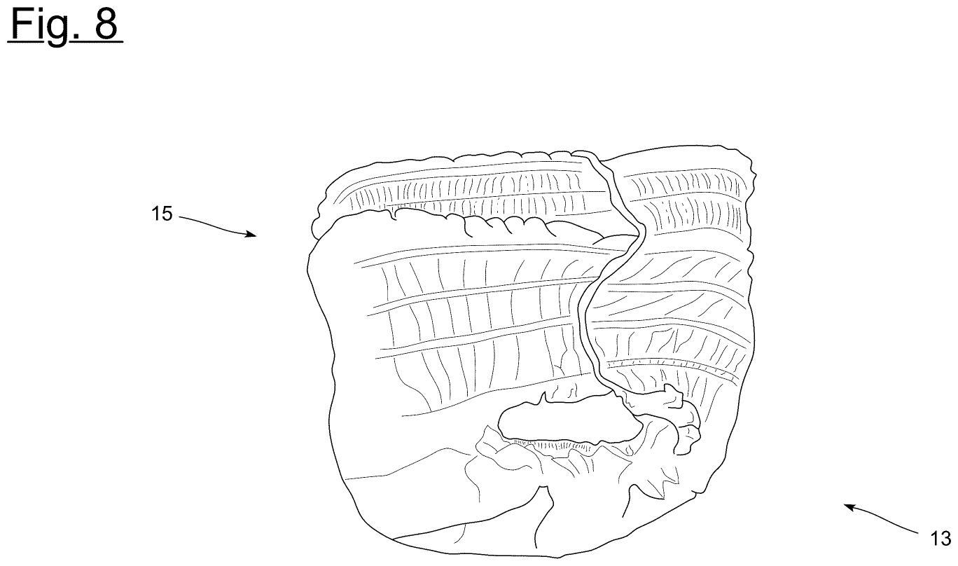

The disposable hygiene article 13 is shown in FIG. 8, wherein the front and the back waistband region 15 are connected with one another.

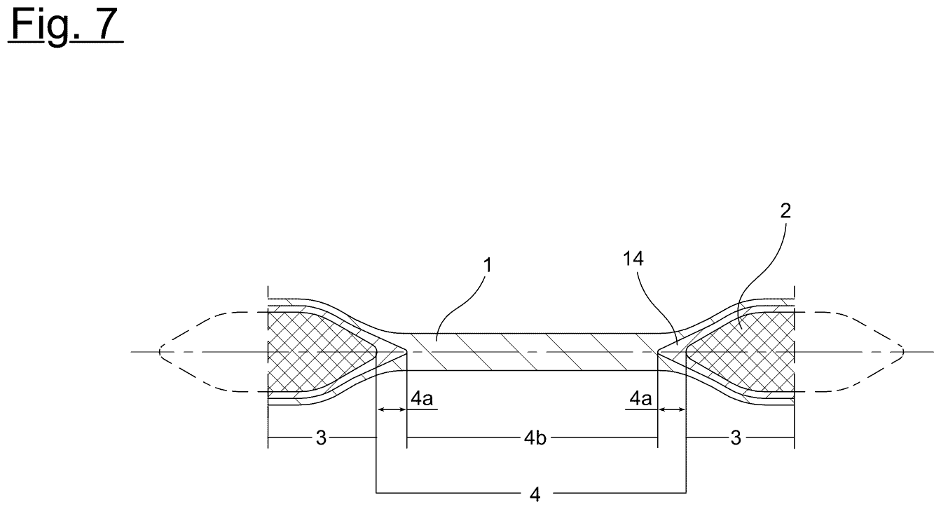

FIG. 7 shows a further embodiment of the co-extruded laminate film having the first, second, and third polymer material 1, 2, 14. The third polymer material 14 is structured in the form of strips, but these are wider than the strips of the second polymer material. In this way it is possible, for example, to divide the second functional section 4 into a partial region 4a that is still elastic or, as described above, semi-elastic, and a non-elastic partial region 4b. The disposable hygiene article 13 shown in FIG. 8 can then have a continuous cutout of the co-extruded laminate film as an outer layer, wherein then the non-elastic partial region holds the absorbent core (see FIG. 6).

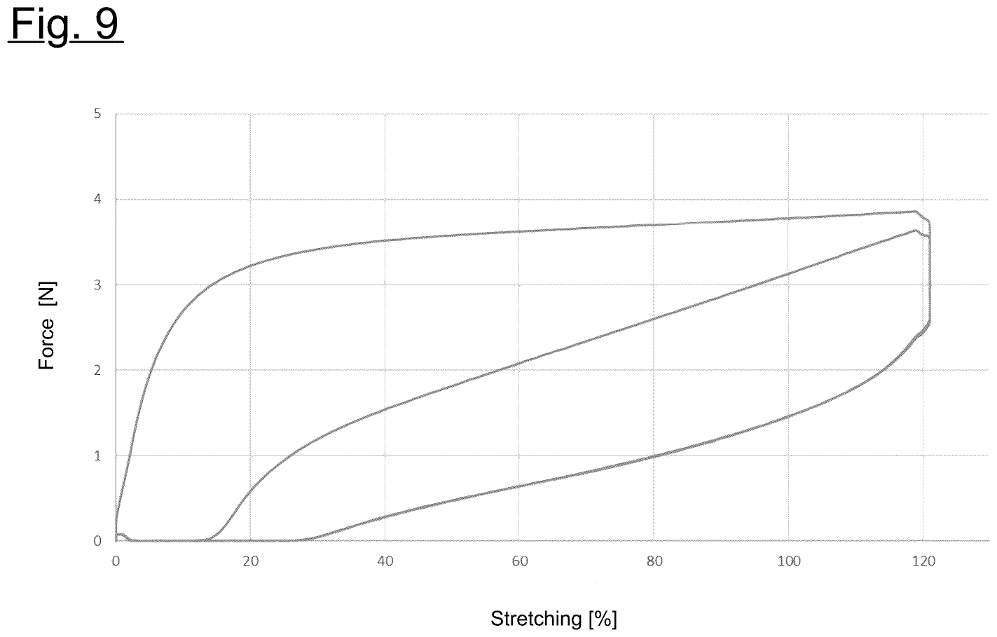

FIG. 9 shows, as an example, a force/stretching diagram for the first elastic functional section of the fifth exemplary embodiment according to Table 1, in the hysteresis measurement that has been described. The uppermost curve shows the behavior during the first stretching. The center curve shows the behavior during the second stretching. Essentially the same behavior occurs for the recovery of the material for both cycles, so that the corresponding lower curves lie on top of one another.

Although only a few embodiments of the present invention have been shown and described, it is to be understood that many changes and modifications may be made thereunto without departing from the spirit and scope of the invention.

What is claimed is:

1. A cover layer for a disposable hygiene article, comprising a co-extruded laminate film having a first full-area polymer material and a second, elastic polymer material, the second elastic polymer material being arranged only in certain regions of the co-extruded laminate and being in the form of at least one strip, so that the co-extruded laminate forms at least one elastic first functional section having the second, elastic polymer material, and at least one second functional section without the second, elastic polymer material, wherein the co-extruded laminate film has an increased thickness on the at least one strip as compared to the at least one second functional section.

2. The cover layer according to claim 1, wherein the at least one second functional section is non-elastic.

3. The cover layer according to claim 1, wherein a ratio of a maximum film thickness (dₘₐₓ) determined on the first functional section to a minimum film thickness (dₘᵢₙ) determined on the second functional section is at least 1.4:1 and/or wherein the ratio of a maximum total thickness (a₂) of the first polymer material, determined on the second functional section, to a minimum total thickness (a₁) of the first polymer material, determined on the first functional section, is at least 2:1.

4. The cover layer according to claim 1, wherein the second, elastic polymer material is arranged in the form of precisely two strips that lie adjacent to longitudinal edges of the laminate film in a longitudinal direction, wherein in a center region that runs along a longitudinal direction, the strips are removed, at least in part, by means of leg cutouts on sides of the laminate film.

5. The cover layer according to claim 4, wherein the strips form elastic ears that are integrated into the cover layer, in at least one waist region that follows the center region in the longitudinal direction.

6. The cover layer according to claim 1, wherein the co-extruded laminate film is laminated to a nonwoven, at least in certain regions.

7. The cover layer according to claim 1, wherein the co-extruded laminate film is pre-stretched, at least in certain regions.

8. A co-extruded laminate film according to claim 1, wherein the at least one strip has a width between 1 cm and 12 cm.

9. The co-extruded laminate film according to claim 1, wherein at least two elastic first functional regions (3) in the form of strips are provided, wherein the distance between the strips is between 3 cm and 24 cm.

10. A disposable hygiene article having a cover layer according to claim 1.

11. The disposable hygiene article according to claim 10, wherein the cover layer is a water-tight outer layer or a partial region of a water-tight outer layer, and further comprising a water-permeable inner layer and an absorbent core arranged between the outer layer and the inner layer.

12. The disposable hygiene article according to claim 10, in the form of a diaper, wherein the cover layer forms an integrated elastic waistband or diaper ear in the at least one elastic section of the co-extruded laminate film, in a waist region.

13. The disposable hygiene article according to claim 12, wherein the cover layer forms two diaper ears, on each of which a closure means with a hook-and-loop closure is arranged.

14. The disposable hygiene article according to claim 10, wherein the at least one strip runs along a longitudinal direction of a diaper body, from a front waistband region over a center region to a back waistband region.

15. The disposable hygiene article according to claim 14, wherein the at least one strip comprises two strips that run adjacent to longitudinal edges, along the longitudinal direction, and wherein the strips are removed, at least in part, in the center region.

16. The disposable hygiene article according to claim 10, wherein the at least one strip runs along a transverse direction of a diaper body, in a front waistband region and/or in a rear waistband region.
